(19) United States
(12) Patent Application Publication
(10) Pub. No.: US 2024/0285541 A1
(43) Pub. Date: Aug. 29, 2024

(54) SHELF STABLE ORGANIC NUCLEOTIDE COMPOSITIONS AND METHODS OF MANUFACTURING THE SAME

(71) Applicant: Glanbia Nutritionals, Inc., Twin Fals, ID (US)

(72) Inventors: Rhitika Poudel, Twin Falls, ID (US); Brent Petersen, Twin Falls, ID (US); Corbin Hohl, Twin Falls, ID (US)

(73) Assignee: Glanbia Nutritionals, Inc., Twin Falls, ID (US)

(21) Appl. No.: 18/587,819

(22) Filed: Feb. 26, 2024

Related U.S. Application Data
(60) Provisional application No. 63/486,723, filed on Feb. 24, 2023.
(60) Provisional application No. 63/502,291, filed on May 15, 2023.

(51) Int. Cl. A61K 9/50; A61K 9/10; A61K 31/7076; A61K 47/02; A61K 47/10; A61K 47/44

(52) U.S. Cl. CPC A61K 9/5036; A61K 9/10; A61K 9/5073; A61K 31/7076; A61K 47/02; A61K 47/10; A61K 47/44

(57) ABSTRACT

Compositions and methods for increasing the solubility and stability of organic nucleotide materials via microencapsulation techniques. A method for making a shelf-stable organic nucleotide compound in a more water-soluble form, the higher-solubility form being provided in liquid and/or powder. The method utilizes a colloidal dispersion solution comprising water, polysaccharides, organic nucleotide and in some cases surfactants. A method for making shelf-stable composition comprising at least one organic nucleotide composition may include combining water, surfactant, polysaccharide, and ATP to form an intermixed colloidal dispersion. The method may include heating the admixed dispersion and running the dispersion under medium shear, wherein the colloidal dispersion provides improved shelf stability to the target nucleotide in water-soluble form.

### TECHNICAL FIELD

The disclosure relates generally to compositions and methods for increasing the solubility and stability of organic nucleotide materials via microencapsulation techniques. More specifically, the disclosure relates to methods for formulating products comprising Adenosine 5′-Triphosphate (ATP) Disodium in a shelf stable environment, even after high heat processing under neutral and acidic conditions.

### BACKGROUND

Adenosine triphosphate (ATP) is a molecule that plays a critical role in providing energy for various biological processes in cells. ATP is a nucleotide composed of an adenine nitrogenous base, a ribose sugar, and three phosphate groups. ATP plays a functional roll within cellular metabolism, neurotransmission, muscle contraction, cardiac function, platelet formation, vasodilation, and liver glycogen metabolism.

In recent years, researchers have become interested in the instability of ATP, particularly in solutions where it is stored on transported. When ATP becomes unstable, it can break down and lose its ability to provide energy for cellular processes. Specifically, like many organic compounds with biological benefits, ATP is not completely stable or soluble in water after exposure to heat and/or acid. In solid form, ATP disodium salt is very stable, showing little to no signs of degradation under normal conditions except when exposed to high moisture and heat. However, in aqueous solution, ATP degrades via hydrolyzation into adenosine diphosphate (ADP) and adenosine monophosphate (AMP). The solubility of ATP may be increased at lower pH and with higher temperatures, but these environmental parameters significantly decrease the stability of ATP.

Studies have shown that oral administration of ATP can lead to increased muscle mass, strength, and endurance. Thus, it is desirable to produce shelf stable ATP compositions that are made only with ingredients that are Generally Recognized As Safe (GRAS). Specifically, sports drinks beverages may be formulated with supplemental ATP to enable increased athletic performance and aid in muscle recovery. However, traditional foods and beverages comprising ATP are not shelf stable for a significant duration of time because the ATP naturally hydrolyzes to ADP or AMP.

However, traditional methods for increasing the shelf stability of ATP are made with components that are not regarded as food safe. Complex coacervation methods and architectures have been evaluated and developed to provide a protective barrier for ATP that is specific to certain pH ranges.

For example, U.S. Patent Publication Number 2009/0143348 ('348 publication) describes a method for producing a biocompatible polysaccharide gel composition with sustained release properties. The methods described in the '348 publication utilize several ingredients that are not suitable for food products and are only suitable for biomedical or pharmaceutical applications. Further for example, U.S. Pat. No. 9,661,870 ('870 patent) describes a nanogel comprised of polysaccharides but does not reference any other polysaccharides outside of soluble soy polysaccharides. Additionally, the '870 patent does not describe wherein the shelf stability of organic nucleotides is increased through microencapsulation with food grade products.

In view of the foregoing, disclosed herein are compositions and methods for increasing the solubility and stability of organic nucleotide materials via microencapsulation techniques.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1 is a schematic illustration of a chemical formulation for adenosine triphosphate (ATP);

FIG. 2 is a schematic illustration of a chemical formulation for adenosine triphosphate (ATP) disodium salt;

FIG. 3 is a schematic illustration of a chemical reaction representing the hydrolyzation of ATP to adenosine diphosphate (ADP);

FIG. 4 is a schematic block diagram of a process flow for generating a microencapsulation formulation for increasing the shelf stability of organic nucleotides;

FIG. 5 is a graphical representation illustrating the unexpectedly good results for increasing the shelf stability of organic nucleotides through food-grade microencapsulation as described in FIG. 4;

FIG. 6 is a schematic block diagram of a process flow for generating a microencapsulation formulation for increasing the shelf stability of organic nucleotides;

FIG. 7 is a graphical representation illustrating the unexpectedly good results for increasing the shelf stability of organic nucleotides through food-grade microencapsulation as described in FIG. 6;

FIG. 8 is a schematic illustration of an example polysaccharide and surfactant coacervation microencapsulate for protecting organic nucleotides;

FIG. 9 is a schematic illustration of an example polysaccharide concervation microencapsulate for protecting organic nucleotides; and

FIG. 10 is a schematic flow diagram of a method for producing a composition for increasing the shelf stability of organic nucleotides through microencapsulation.

### DETAILED DESCRIPTION

Disclosed herein are compositions, systems, and methods for increasing the solubility and stability of organic nucleotide materials via microencapsulation techniques. More specifically, the disclosure relates to compositions and methods for lengthening the shelf stability of adenosine triphosphate (ATP) even after high heat processing under neutral and acidic conditions. Disclosed herein are compositions and methods relating to a coacervated microgel encapsulation and technique to protect water soluble organic nucleotide compositions from harsh conditions such as pH and high temperature processing.

Specifically described herein are compositions and methods for preparing a microencapsulation for bioactive molecules, such as one or more of one or more of ATP, ATP disodium salt, L-theanine, L-glutamine, taurine, β-alanine, or carnitine. The microencapsulates described herein increase the shelf stability of organic nucleotides. Specifically, the microencapsulates described herein are effective in preventing ATP from hydrolyzing into adenosine diphosphate (ADP) or adenosine monophosphate (AMP) in the presence of water. The compositions and methods described herein utilize only food grade materials that are Generally Recognized as Safe (GRAS), such that the microencapsulated organic nucleotides described herein may be utilized in food products, such as beverages, gelatins, and other foods.

The composition described herein are prepared for human and animal consumption. Food-grade ingredients are often susceptible to degradation under high temperature processing conditions, including, for example, hot fill processing, high temperature short time (HTST) pasteurization, or ultra-high temperature (UHT) processing. However, the microencapsulation methods and compositions described herein are provided to withstand high temperature processing and extreme pH conditions while still using only food-grade ingredients.

Because ATP serves as the primary source of energy for cells within the body, and because it plays a crucial role in many physiological processes, including muscle contraction, nerve impulses, and metabolism, it can be desirable to ingest supplemental ATP. ATP supplementation is linked to improved athletic performance, increased muscle mass, improved recovery, enhanced cognitive function, and improved cardiovascular health. ATP supplementation helps to increase energy levels, delay fatigue, and improve endurance during high-intensity exercise. This is likely due to ATP being the primary source of energy needed for muscle contraction. Additionally, studies suggest that ATP supplementation leads to increase muscle mass and strength by promoting the synthesis of muscle proteins. ATP supplementation is also known to aid in muscle recovery by reducing muscle damage and inflammation.

The potential benefits of oral ATP supplementation are demonstrated in U.S. Pat. No. 7,629,329 B2, which discloses that oral administration of ATP can lead to improvements to muscle mass and strength. Studies show that oral administration of ATP disodium salt can improve ATP blood plasma concentrations for up to two hours post-administration. In another study, Wingate endurance testing methodology highlighted a statistical improvement in muscular recovery in the 90-120-minute range in subjects who received ATP disodium salt in tablet form. In other research, the ATP content of red blood cells (RBC) and plasma was shown to be comparable between intravenous administration and oral administration, while dose dependent. Ultimately, oral ingestion of ATP is surmised to function via increases to blood flow, increases to muscular excitability, and anabolic signaling.

Some compositions described herein rely on microencapsulation to protect organic nucleotides from degradation due to hydrolysis, extreme pH, or high-temperature processing. The systems and methods described herein demonstrate an effective means of preparing a self-assembled delivery system targeting organic nucleotides such as ATP. The methods and compositions described herein decrease the chemical degradation of organic nucleotides within extreme pH and high temperature conditions. The microencapsulated components described herein are prepared for applications in beverages (including acidic and neutral beverages), ready-to-mix powders, gels, gummies, jellybeans, nutritional bars, functional foods, tablets, capsules, intravenous fluids, intramuscular fluids, films, and others.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Before the structures, systems, methods, and compositions for increasing the solubility and stability of organic nucleotide materials via microencapsulation techniques are disclosed and described, it is to be understood that this disclosure is not limited to the structures, configurations, process steps, and materials disclosed herein as such structures, configurations, process steps, and materials may vary somewhat. It is also to be understood that the terminology employed herein is used for the purpose of describing embodiments only and is not intended to be limiting since the scope of the disclosure will be limited only by the appended claims, if any, and equivalents thereof.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

As used herein, “effective amount” means an amount of an ingredient or a component of the product that is nontoxic, but sufficient to provide the desired effect and performance at a reasonable benefit/risk ratio attending any dietary supplement or product.

Unless defined otherwise, all technical and scientific terms used herein have the same meanings as commonly understood by one of ordinary skill in the art to which this disclosure pertains and belongs.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to embodiments are not restricted to only those embodiments in which they are described. For example, an element described in reference to one embodiment or figure, may be alternatively included in another embodiment or figure regardless of whether those elements are shown or described in another embodiment or figure. In other words, elements in the figures may be interchangeable between various embodiments disclosed herein, whether shown or not.

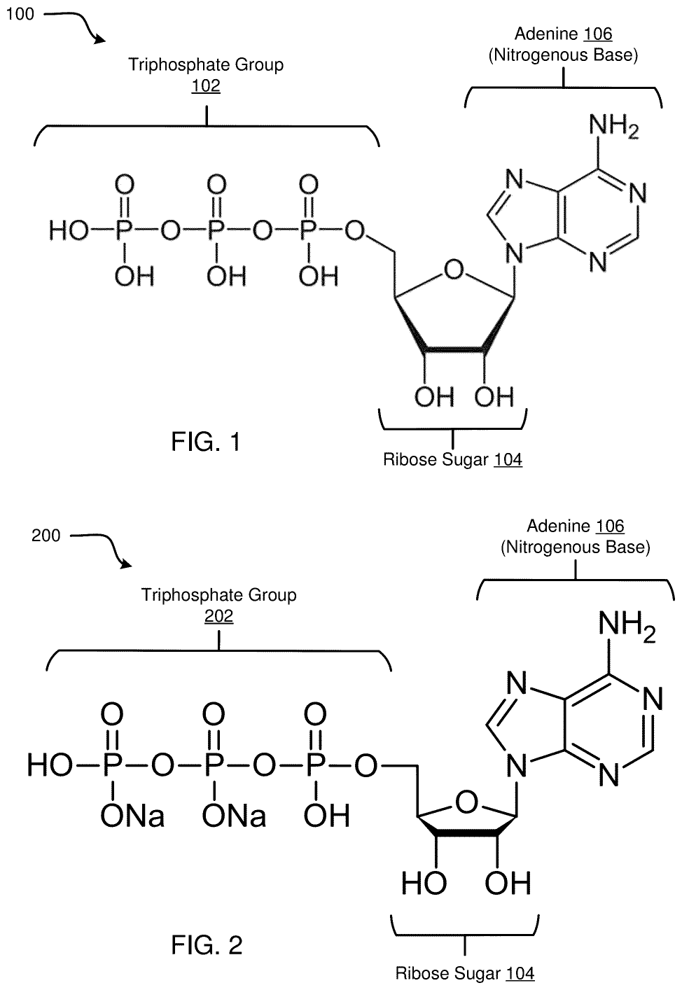

Referring now to the figures, FIGS. 1 and 2 are chemical formulas of adenosine triphosphate (ATP). FIG. 1 illustrates ATP 100 with hydroxyl functional groups (having the chemical formula OH and comprising one oxygen atom covalently bonded to one hydrogen atom). FIG. 2 illustrates ATP disodium salt 200, which includes two sodium molecules attracted to two oxygen molecules of the triphosphate group. As discussed herein, each of ATP 100 and ATP disodium salt 200 may be referred to as ATP 100, 200.

ATP 100 and ATP disodium salt 200 each include a triphosphate group 102. In the hydroxyl configuration illustrated in FIG. 1, the triphosphate group 102 include four hydroxyl groups. In the disodium salt configuration illustrated in FIG. 2, the triphosphate group 202 include two hydroxyl groups and two sodium anions. ATP 100 and ATP disodium salt 200 further include a ribose sugar 104 and an adenine 106 molecule.

ATP 100 and ATP disodium salt 200 consists of the adenine 106 attached by the 9-nitrogen atom to the 1′ carbon atom of the ribose 104 which in turn is attached at the 5′ carbon atom of the sugar to a triphosphate group 102, 202. In its many reactions related to metabolism, the adenine 106 and ribose sugar 104 groups remain unchanged, but the triphosphate group 102, 202 is converted to diphosphate to generate adenosine diphosphate (ADP) or to monophosphate group to generate adenosine monophosphate (AMP). The three phosphoryl groups are labeled as alpha, beta, and for the terminal phosphate, gamma. In neutral solution, ionized ATP 100 exists mostly as ATP⁴⁻, with a small proportion of ATP³⁻.

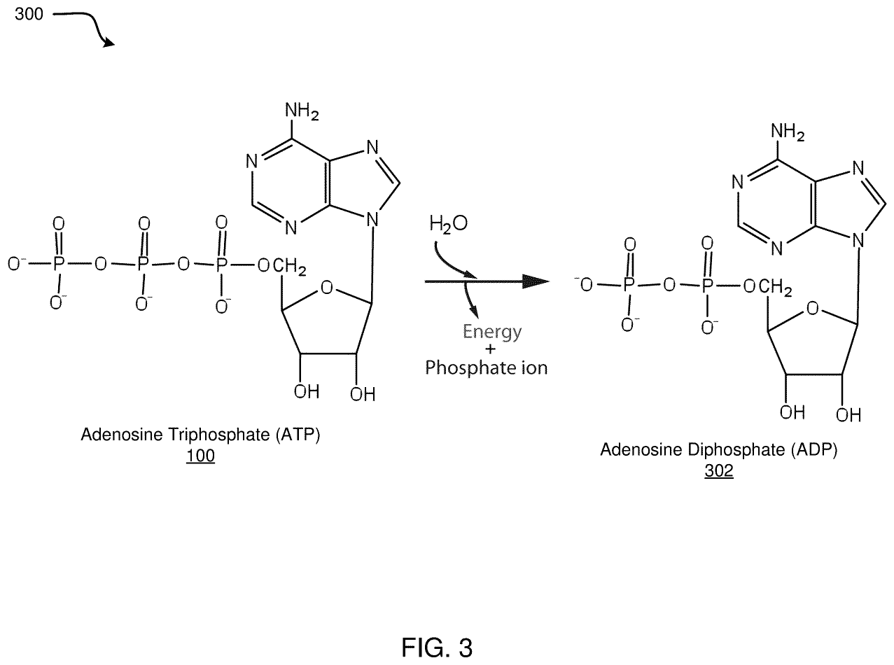

ATP 100 is an organic compound that provides energy to drive and support many processes in living cells, such as muscle contraction, nerve impulse propagation, condensate dissolution, and chemical synthesis. Found in all known forms of life, ATP 100 is often referred to as the “molecular unit of currency” of intracellular energy transfer. When consumed in metabolic processes, ATP 100 converts either to adenosine diphosphate (ADP, see reaction illustrated in FIG. 3) or to adenosine monophosphate (AMP). Other processes regenerate ATP 100. The human body recycles its own body weight equivalent in ATP 100 each day. ATP 100 serves as a precursor to DNA (deoxyribonucleic acid) and RNA (ribonucleic acid) and is used as a coenzyme. From the perspective of biochemistry, ATP 100 is classified as a nucleoside triphosphate, which indicates that it consists of three components, including a nitrogenous base (adenine 106), a sugar (ribose 104), and the triphosphate group 102.

ATP disodium salt 200 is a form of ATP that is used as a coenzyme in cells. ATP disodium salt 200 is used in many cellular processes, including respiration, biosynthetic reactions, motility, and cell division. ATP 100, 200 is a substrate of many kinases involved in cellular signaling and of adenylate cyclase that produce the second messenger cAMP.

Salts of ATP 100, such as ATP disodium salt 200, can be isolated as colorless solids. ATP 100 is stable in aqueous solutions between pH 6.8 and 7.4, in the absence of catalysts. At more extreme pHs, ATP 100 rapidly hydrolyses to ADP and phosphate. Living cells maintain the ratio of ATP 100 to ADP at a point ten order of magnitude from equilibrium, with ATP 100 concentrations fivefold higher than the concentration of ADP.

Described herein are compositions and methods for microencapsulation of an active bioagent, which may specifically include an organic nucleotide such as ATP 100, 200. The active bioagents described herein may include ATP 100, 200, and may alternatively include one or more of L-theanine, L-glutamine, taurine, β-alanine, and carnitine. The microencapsulate structures described herein include an inner core composed of a polysaccharide matrix intermingled with the bioactive molecule in a coacervate phase. The polysaccharide matrix may further be coated with a surfactant and polysaccharide matrix shell. In this microencapsulated embodiments, the bioactive molecule intermixed with the polysaccharide matrix may include one or more of ATP 100, ATP disodium salt 200, L-theanine, L-glutamine, taurine, β-alanine, or carnitine.

FIG. 3 illustrates a chemical reaction 300 for the hydrolysis of ATP 100 into adenosine diphosphate (ADP) 302. The reaction 300 releases 20.5 KJ/mol of enthalpy. In the context of biochemical reactions, the P—O—P bonds are frequently referred to as high-energy bonds. As shown in FIG. 3, the reaction 300 requires the introduction of water (H₂O) and results in the release of energy, one phosphate ion, and ADP 302.

The compositions and methods described herein result in increased stability of aqueous solutions comprising ATP 100. The compositions described herein are manufactured using the disclosed methods with ingredients such as polysaccharides and surfactants, all being Generally Recognized As Safe (GRAS), thereby increasing the stability of bioactive composition under processing conditions. In some implementations described herein, the shelf stability of ATP 100 is increased by manufacturing coacervate microencapsulates comprising at least one water soluble bioactive composition. The microencapsulates described herein prevent the hydrolysis of ATP 100 to ADP 302 when ATP 100 interacts with water.

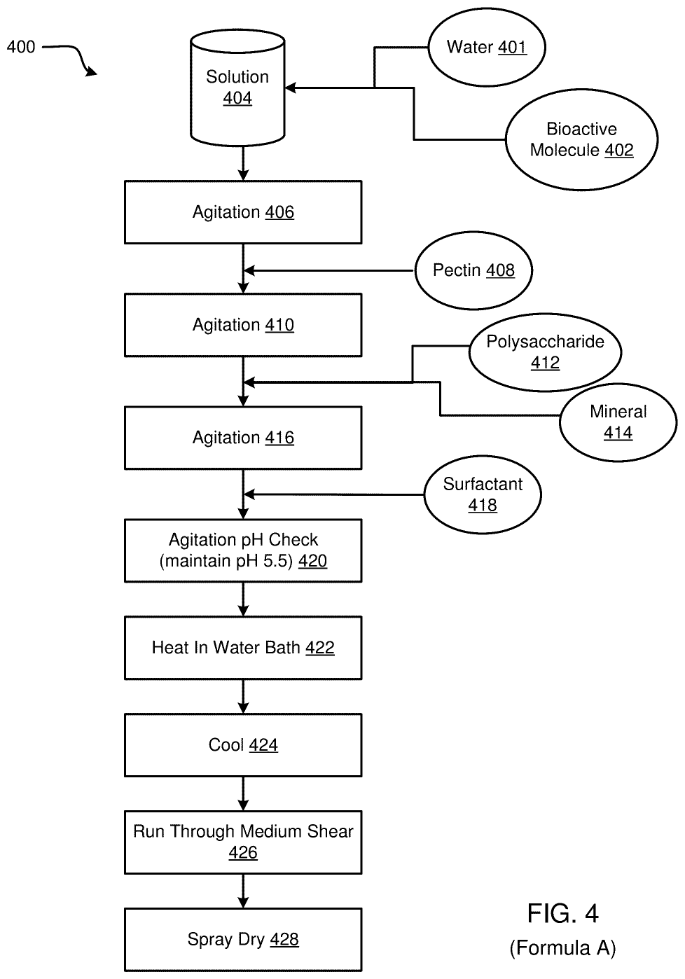

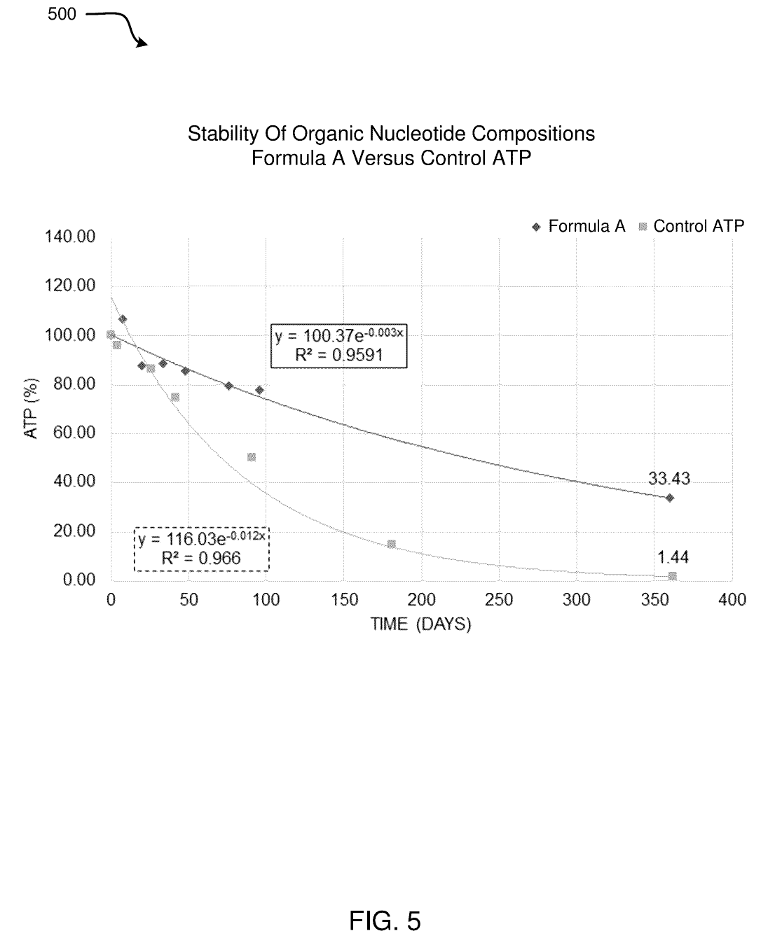

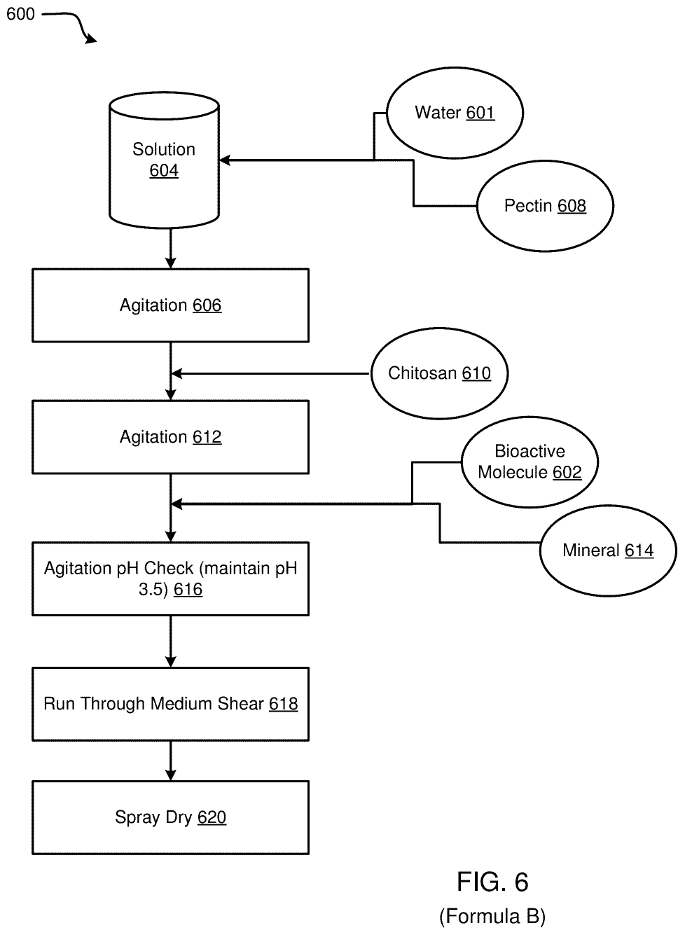

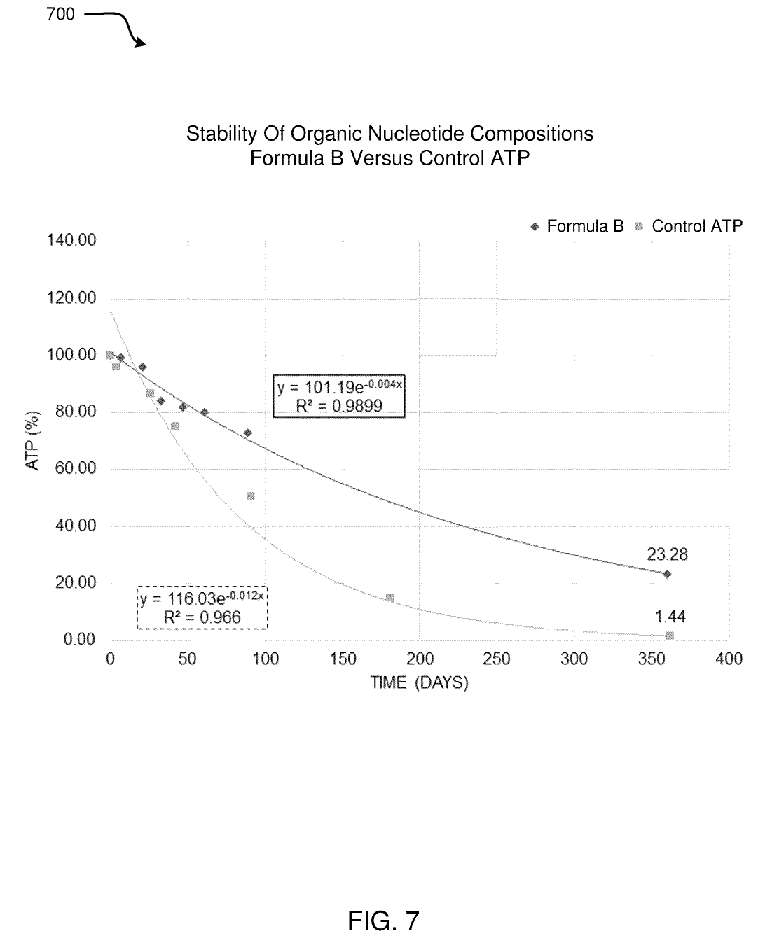

FIGS. 4-7 pertain to compositions and methods described herein for increasing the shelf stability of ATP 100, 200. FIGS. 4 and 6 are schematic flow chart diagrams of methods 400, 600 for generating compositions that exhibit increased stability of ATP 100, 200 in aqueous solution. FIGS. 5 and 7 are graphical illustrations of the unexpectedly good results achieved through the methods 400, 600 described in FIGS. 4 and 6. Specifically, FIGS. 4-5 describe compositions, methods, and results for a first formula described herein, which may be referred to herein as Formula A. FIGS. 6-7 describe compositions, methods, and results for a second formula described herein, which may be referred to herein as Formula B.

Referring now to FIG. 4, the method 400 includes preparing a solution 404 comprising water 401 and a bioactive molecule 402. The bioactive molecule 402 may include an organic nucleotide and may specifically include one or more of ATP 100, ATP disodium salt 200, L-theanine, L-glutamine, taurine, β-alanine, or carnitine. In some cases, the water 401 is heated to a temperature of about 40° to about 60° Celsius prior to adding the bioactive molecule 402 to generate the solution 404. The method 400 continues and the solution 404 is agitated at 406, which may include stirring or shaking the solution 404. The mechanical energy added to the system at 406 aids in mixing the water 401 and the bioactive molecule 402 to form a homogenous solution.

Pectin 408 is then added to the solution 404, and the solution 404 (comprising each of the water 401, the bioactive molecule 402, and the pectin 408) is again agitated at 410. Pectin 408 is composed of complex polysaccharides that are present in the primary cell wells of a plant and are abundant in the green parts of terrestrial plants. The principal chemical component of pectin 408 is galacturonic acid (a sugar acid derived from galactose). Commercially produced pectin 408 is produced from citrus fruits for use as an edible gelling agent, and is often used as a thickener in cooking, baking, and medicine manufacturing.

The method 400 continues and a polysaccharide 412 and mineral 414 are added to the solution 404 (which now comprises each of the water 401, the bioactive molecule 402, the pectin 408, the polysaccharide 412, and the mineral 414). Again, the solution 404 is agitated at 416.

The polysaccharide 412 is a carbohydrate and may specifically include a long-chain polymeric carbohydrate composed of monosaccharide units bound together by glycosidic linkages. The polysaccharide 412 may be composed of simple carbohydrates referred to as monosaccharides, which have the general formula of (CH₂O)ₙ, where n is equal to three or more. Examples of monosaccharides include glucose, fructose, and glyceraldehyde. The polysaccharide 412 may have the general formula of Cₓ(H₂O)_{y} where x is typically a substantial number between 200 and 2500. When the repeating units in the polymer backbone include six-carbon monosaccharides (often the case), the general formula of the polysaccharide 4412 simplifies to (C₆H₁₀O₅)ₙ, where n is typically between 40 and 3000.

The polysaccharide 412 may specifically include one or more of cellulose, starch, glycogen, chitin, pectin, or chitosan. Cellulose is a polysaccharide consisting of a linear chain of linked D-glucose units. Starch is a polysaccharide carbohydrate consisting of a large number of glucose monosaccharide units joined together by glycosidic bonds. Glycogen is a branched polymer of glucose that is mainly produced in liver and muscle cells, and functions as secondary long term energy storage in animal cells. Chitin is a polymer of nitrogen-containing polysaccharides rendering a tough protective covering or structural support in certain organisms.

The mineral 414 is an inorganic compound that may specifically be selected to effectuate a desired result in a human or animal that will consume the resultant solution 404. The mineral 414 may additionally be selected to increase the long-term preservation of the solution 404. In an example implementation, the mineral 414 includes one or more of calcium, magnesium, chloride, phosphate, potassium, or sodium.

The method 400 continues and a surfactant 418 is added to the solution 404 (which now includes each of the water 401, bioactive molecule 402, pectin 408, polysaccharide 412, mineral 414, and surfactant 418). The solution 404 is then agitated again at 420. Additionally, the pH of the solution is verified at step 420, and the pH of the solution is brought to and maintained at about 5.2 to about 5.8 and may specifically be maintained at 5.5.

The surfactant 418 is a chemical compound that decreases the surface tension or interfacial tension between two liquids, a liquid and a gas, or a liquid and a solid. The surfactant 418 may include one or more of an emulsifier, wetting agent, foaming agent, or dispersant. Specifically, the surfactant 418 may include a food grade surfactant including one or more of an ethoxylated fatty amine, alkylphenol ethoxylate-based surfactant (non-ionic), alcohol ethoxylate-based surfactant (non-ionic), silicone-based surfactant, oil, alkyl glycoside, carrageenan (carbohydrate), cholesterol, lanolin, lecithin, monoglyceride (fatty acid), phytosterol, protein, or saponin extract.

The surfactant 418 may specifically include a lecithin extracted from one or more of egg yolks, marine foods, soybeans, milk, rapeseed, cottonseed, or sunflower oil. The surfactant 418 may specifically include one or more of polyethylene glycol (PEG), propylene glycol, or propanediol.

In the compositions described herein, the surfactant 418 may specifically be selected to reduce s surface tension between a liquid phase and a solid phase. For example, the solid phase may include ATP disodium salt 200 and the liquid phase may include an admixed colloidal dispersion slurry.

The method 400 continues and the solution 404 is heated in a water bath at 422. In some implementations, the solution 404 is heated to a temperature of about 65° Celsius to about 80° Celsius and may specifically be heated to about 72° Celsius. The solution 404 is maintained at the heated temperature for a duration of time. Specifically, the solution 404 may be held at the increased temperature for about 20 minutes to about 50 minutes.

The method 400 continues and the solution 404 is cooled down at 424 to a temperature of about 15° Celsius to about 30° Celsius and may specifically be cooled to about 22° Celsius. The solution 404 is then processed through a medium shear at 426 for several minutes and may specifically be processed through the shear from about 1 minute to about 5 minutes. The solution 404 is then spray dried at 428, which may include spraying with an inlet temperature of about 210° Celsius to about 250° Celsius, and an outlet temperature of about 80° Celsius to about 110° Celsius.

FIG. 5 is a graphical representation 500 of the unexpectedly good results achieved through the method and composition described in connection with FIG. 4. Specifically, the graphical representation 500 illustrates a comparison of ATP stability for a composition or product of the disclosure (specifically, Formula A prepared according to method 400) compared against a control ATP product processed under acidic conditions (pH 2.5) and hot-fill conditions. The ATP stability measurements were calculated over three hundred sixty-two (362 days). For the control ATP, pH 2.8 (Δ), y=116.03e^{-0.012x}, R²=0.966. For the encapsulated ATP Formula A, pH 2.8 (Δ), y=100.37e^{-0.003x}, R²=0.9591.

As shown in FIG. 5, Formula A (i.e., the composition prepared according to the method 400 described in FIG. 4) comprised greater quantities of ATP 100, 200 than the control ATP solution. Specifically, Formula A continued to comprise about 33.43 wt % of ATP 100, 200 at the end of the 362 days, compared with the 1.44 wt % of ATP 100, 200 in the control ATP solution.

Referring now to FIG. 6, the method 600 includes preparing a solution 604 comprising water 601 and pectin 608. The pectin 608 may include one or more of any of the pectin 408 compounds discussed in connection with FIG. 4. In some cases, the water 601 is heated to a temperature of about 40° to about 60° Celsius prior to adding the pectin 608 to generate the solution 604. Specifically, the water 601 may be heated to a temperature of about 50° Celsius prior to adding the pectin 608. The method 600 continues and the solution 604 is agitated at 606, which may include stirring or shaking the solution 604. The mechanical energy added to the system at 606 aids in mixing the water 601 and the pectin 608 to form a homogenous solution.

The method 600 continues and chitosan 610 is then added to the solution 604, and then the solution 604 is again agitated at 612. The chitosan 610 is a linear polysaccharide composed of deacetylated units and acetylated units. Chitosan 610 is made by treating the chitin shells of shrimp and other crustaceans with an alkaline substance, such as sodium hydroxide. Chitosan 610 has a number of commercial and biomedical uses.

The method 600 continues and a bioactive molecule 602 and a mineral 614 are added to the solution 604 (which now includes each of the water 601, pectin 608, chitosan 610, bioactive molecule 602, and mineral 614). The mineral 614 may include one or more of any of the minerals 414 described in connection with FIG. 4. Specifically, the mineral 614 is an inorganic compound that may specifically be selected to effectuate a desired result in a human or animal that will consume the resultant solution 604. The mineral 614 may additionally be selected to increase the long-term preservation of the solution 604. In an example implementation, the mineral 614 includes one or more of calcium, magnesium, chloride, phosphate, potassium, or sodium.

The method 600 continues and the solution 604 is agitated and the pH is verified at 616. The pH of the solution is brought to and maintained at about 3.2 to about 3.8 and may specifically be maintained at about 3.5. The method 600 continues and the solution 604 is processed through a medium shear at 618 for several minutes and may specifically be processed through the shear from about 1 minute to about 5 minutes. The solution 604 is then spray dried at 628, which may include spraying with an inlet temperature of about 210° Celsius to about 250° Celsius, and an outlet temperature of about 80° Celsius to about 110° Celsius.

FIG. 7 is a graphical representation 700 of the unexpectedly good results achieved through the method and composition described in connection with FIG. 6. Specifically, the graphical representation 700 illustrates a comparison of ATP stability for a composition or product of the disclosure (specifically, Formula B prepared according to method 600) compared against a control ATP product processed under acidic conditions (pH 2.8) and hot-fill conditions. ATP stability measurements were calculated over three hundred sixty-two (362) days. For ATP disodium salt, pH 2.8 (Δ), y=116.03e^{-0.012x}, R²=0.966. For Encapsulated ATP Formula 8, pH 2.8 (Δ), y=101.19e^{-0.004x}, R²=0.9899.

As shown in FIG. 7, Formula B (i.e., the composition prepared according to the method 600 described in FIG. 6) comprised greater quantities of ATP 100, 200 than the control ATP solution. Specifically, Formula B continued to comprise about 23.28 wt % of ATP 100, 200 at the end of the 362 days, compared with the 1.44 wt % of ATP 100, 200 in the control ATP solution.

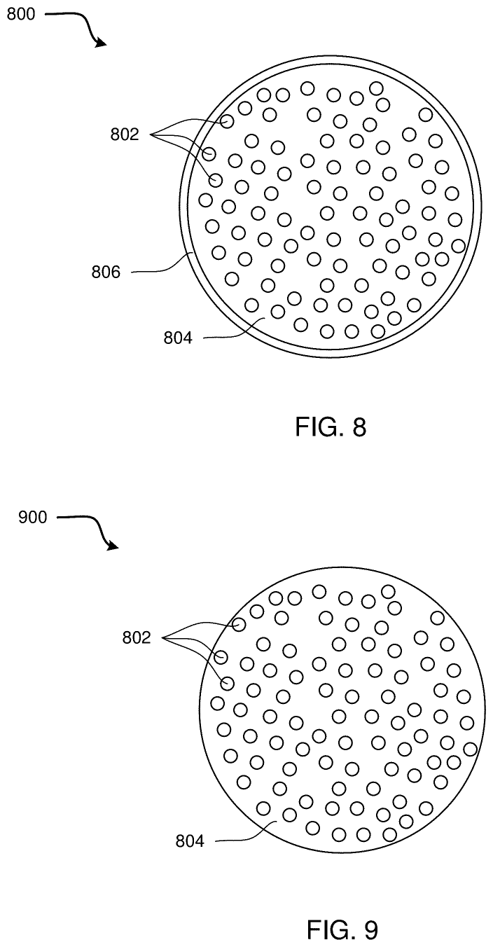

FIGS. 8 and 9 are schematic illustrations of example structures 800, 900 of microencapsulates configured to receive and protective a bioactive molecule 802 such as ATP 100 or ATP disodium salt 200. The microencapsulate structures 800, 900 illustrated in FIGS. 8 and 9 are generated when preparing a solution according to the methods 400, 600 described herein.

The microencapsulate structures 800, 900 protect bioactive molecules 802, such as the bioactive molecules 402, 602 discussed in connection with FIGS. 4 and 6. The bioactive molecules 802 may include one or more of ATP 100, ATP disodium salt 200, L-theanine, L-glutamine, taurine, β-alanine, or carnitine. The structures 800, 900 prevent the degradation of the bioactive molecules 802 in extreme pH and high temperature processing conditions. This enables the bioactive molecules 802 to be used in beverages (including acidic and neutral beverages), ready-to-mix powders, gels, gummies, jellybeans, nutritional bars, functional foods, tablets, capsules, solutions for intravenous administration, solutions for intramuscular administration, films, and so forth.

FIG. 8 is a schematic illustration of a structure 800 of a polysaccharide and surfactant coacervation microencapsulate. The structure 800 includes a shell 806 comprising a surfactant and/or polysaccharide matric. The structure 800 includes a polysaccharide matrix 804 disposed within the shell 806. The structure 800 additionally includes one or more bioactive molecules 806 distributed within the polysaccharide matrix 804.

As illustrated, the shell 806 comprising the surfactant and/or polysaccharide matrix surrounds the polysaccharide matrix 804 that houses or encapsulates the bioactive molecules 802, which may specifically include any of the bioactive molecules 430, 630 discussed in connection with FIGS. 4 and 6. The bioactive molecules 802 may specifically include ATP 100 or ATP disodium salt 200. The methods 400, 600 described herein are implemented to generate a structure 800 to increase the long-term shelf stability of ATP 100, 200 in aqueous solution.

Specifically, the compositions and methods described herein relate to a coacervated microgel-based encapsulation technique that can be used to protect water soluble organic nucleotide compositions from harsh conditions such as pH and high temperature processing. ATP 100, 200 may be used as a water-soluble bioactive molecule 802 that is protected by the polysaccharide matrix 804 and the shell 806. However, other water-soluble bioactive molecules 806, such as L-Theanine, L-Glutamine, Taurine, β-Alanine and Carnitine, may also be disposed within and protected by the polysaccharide matrix 804 and the shell 806.

FIG. 9 is a schematic illustration of a structure 900 of a polysaccharide coacervation microencapsulate. The structure 900 is like the structure 800 described in connection with FIG. 8, but without the surfactant/polysaccharide shell 806. The structure 900 may exhibit increased rigidity when dispersed in an aqueous phase of a polysaccharide matrix.

As shown in FIGS. 8-9, the microencapsulate structure 800, 900 is avocado-like, with an inner core of polysaccharide matrix 804 intermingled with the bioactive molecules 802 in a coacervate phase. The structure 800 may additionally be coated with a surfactant and polysaccharide matrix shell 806 as shown in FIG. 8. In the alternative structure shown in FIG. 9, the surfactant and polysaccharide matrix shell 806 is omitted, with the microencapsulate rigidity increasing and being dispersed in aqueous phase of polysaccharide matrix 804.

The choice of structure (FIG. 8 or 9) is determined based on the selected bioactive molecule(s) 805 physiochemical characteristics, including pKa, solubility, and particle size. Heating, shear mixing, pH, and hydration play important roles in leading to a successful microencapsulation of the bioactive molecule 802. Batch pasteurization may be useful in forming gel microstructures between the polysaccharide matrix 804 and the bioactive molecules 802, alongside the surfactant and polysaccharide shell 806. Typically, in the structures 800, 900 shown in FIGS. 8 and 9, the loading capacity for the bioactive molecule 802 ranges from about 70% to about 90% with ATP 100, 200.

This disclosure offers a unique preparation for water-soluble bioactive molecules 806 utilizing a polysaccharide matrix 804, with or without a surfactant/polysaccharide shell 806. The methods and compositions described herein utilize food grade ingredients targeting use in food product and supplement applications. The loading capacity of the structures 800, 900 for encapsulating and protecting the bioactive molecule 802 ranges from about 72% to 86% when the bioactive molecule 802 comprises ATP 100 or ATP disodium salt 200.

Throughout the methods 400, 600 for generating the microencapsulate structures 800, 900, the pH is maintained depending on matrix constituents. Specifically, the pH is optimized and maintained to ensure optimal bindings and charge manipulation with the use of acidulants or bases. As the intended use of the disclosure is in finished good products, heat treatments for safety are required, with vat or batch pasteurization, high temperature short time pasteurization or suitable safety step is completed on the slurry, prior to spray drying. The mixture is then spray-dried (Tᵢₙₗₑₜ=200° C. and Tₒᵤₜₗₑₜ=100° C., for example).

Compositions described herein can be successfully processed under hot-fill conditions for high-acid beverages, which are often used to kill bacteria and extend shelf life of liquids, providing a unique avenue for usage of organic nucleotide ingredients as listed. Products of the disclosure include bottles of canned colloidal dispersions made by the methods described herein. These solutions may include one or more of added flavors, coloring, acidulants, stabilizers, and so forth, as desired. Alternatively, powdered drink mixes may be prepared according to the methods described herein by spray drying at neutral or acidic pH.

The disclosure also provides organic nucleotides in a form that can be used in dry, semi-moist, and moist food products, and especially those in acidic conditions. The methods described herein target organic nucleotide compounds in a form that is more protected from the effects of water in the environment surrounding the target organic nucleotide compound. This enables distinct advantage for the formulation of aqueous-based products, as well as dry, semi-moist, and moist products which may have higher water activity. In traditional compositions, the higher water activity would reduce the stability of target organic nucleotides such as ATP 100 or ATP disodium salt 200. The compositions described herein can be implemented in a variety of products formulated for human and/or animal consumption, such as breakfast bars, snack bars, protein bars, gummies, dog, and cat food, and/or treats.

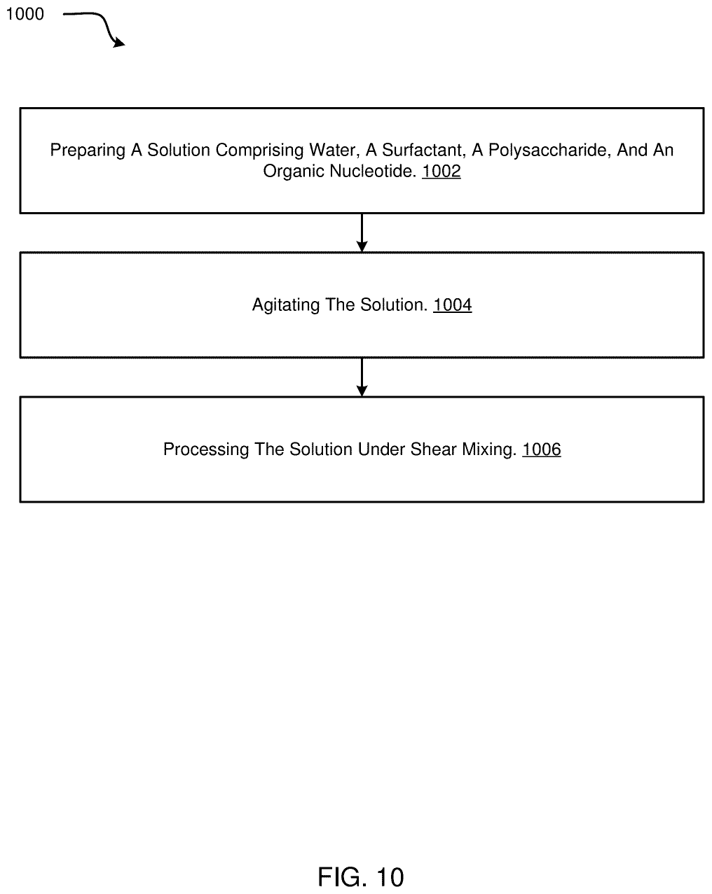

FIG. 10 is a schematic flow chart diagram of a method 1000 for preparing a composition for increasing the shelf stability of an organic nucleotide. The method 1000 includes preparing a solution at 1002 that includes water, a surfactant, a polysaccharide, and an organic nucleotide. The water may include purified or non-purified water. The surfactant may include any of the surfactants described herein, and specifically those surfactants 418 described in connection with FIG. 4. The polysaccharide may include any of the polysaccharides described herein, and specifically those polysaccharides 412 described in connection with FIG. 4. The organic nucleotide may include any organic nucleotide and may specifically include ATP 100 and/or ATP disodium salt 200.

The method 1000 continues and the solution is agitated at 1004. The solution may be agitated over time while the water, surfactant, polysaccharide, and/or organic nucleotide are added to the solution. As shown in FIGS. 4 and 6, the solution may be continuously agitated while various components of the solution are added over time.

The method 1000 continues and the solution is processed under shear mixing at 1006. In specific implementation, the solution is processed under medium shear. Shear mixing includes dispersing or transporting one phase or ingredient (liquid, solid, gas) into a main continuous phase (liquid). The shear mixing process may include causing a rotor or impeller, together with a stationary component referred to as a stator, or an array of rotors and stators. The shear mixing process may be performed within a tank comprising the solution, or within a pipe through which the solution passes, to create shear.

The process of shearing the solution at 1006 may include processing the solution under low shear, medium shear, or high shear. In most implementations, the solution is processed under medium shear mixing at 1006. Medium shear mixing provides moderate shear and flow to effectively blend and disperse the ingredients. Radial and axial flow turbines may be implemented to mix the water, surfactant, polysaccharide, and organic nucleotide ingredients.

### EXAMPLES

The following examples pertain to further embodiments.

The following table is representative of various weight percentages of ingredients used as part of an example composition of the disclosure. As described herein, certain weight percentages are given as in colloidal dispersion. This indicates the concentration of an ingredient in a colloidal dispersion prior to being dehydrated into a dry powder form. A “colloidal dispersion” is a system in which particles of colloidal size (e.g., from about 1 nm to about 1 μm) are dispersed within a continuous phase of a different composition. A “coacervate” is a phenomenon in which a colloidal solution is separated into colloid-rich and colloid-poor phases. Further as described herein, the phrase “as in powder” indicates the concentration of an ingredient in a dried (e.g., dehydrated or spray dried) powder.

| Ingredient Used in | Weight % in Colloidal | |
|---|---|---|
| Formulation | Dispersion | Weight % in Dry Powder |
| Water | About 70 wt % to about 90 wt % | N/A |
| Adenosine | About 10 wt % to about 18 wt % | About 50 wt % to about 90 wt % |
| Triphosphate Disodium | | |
| Polysaccharide Matrix | About 0.5 wt % to about 3.5 | About 2.5 wt % to about 17.7 |
| | wt % | wt % |
| Surfactant Matrix | About 0.4 wt % to about 3.5 | About 2 wt % to about 17.7 |
| | wt % | wt % |
| Mineral Matrix | About 0.1 wt % to about 1.0 | About 0.5 wt % to about 5 wt % |
| | wt % | |

The following table is representative of various weight percentages of ingredients used as part of an example composition of the disclosure.

| Ingredient Used in | Weight % in Colloidal | |
|---|---|---|
| Formulation | Dispersion | Weight % in Dry Powder |
| Water | About 70 wt % to about 90 wt % | N/A |
| Adenosine | About 10 wt % to about 17 wt % | About 56 wt % to about 94 wt % |
| Triphosphate Disodium | | |
| Polysaccharide Matrix | About 0.5 wt % to about 3.5 | About 2.7 wt % to about 19.4 |
| | wt % | wt % |
| Mineral Matrix | About 0.1 wt % to about 1.0 | About 0.5 wt % to about 5.5 |
| | wt % | wt % |

According to one or more embodiments of the disclosure, a composition may include a combination of all or some, but not all, of the following ingredients: water; adenosine triphosphate; adenosine triphosphate disodium salt; pectin; chitosan; propylene glycol; calcium chloride; polysaccharide having a general formula of (CH₂O)ₙ; polysaccharide having a general formula of Cₓ(H₂O)_{y}; polysaccharide having a general formula of (C₆H₁₀O₅)ₙ; cellulose; starch; glycogen; chitin; calcium; magnesium; chloride; phosphate; potassium; sodium; ethoxylated fatty amine; alkylphenol ethoxylate-based surfactant; alcohol ethoxylate-based surfactant; silicone-based surfactant; alkyl glycoside; carrageenan; cholesterol; lanolin; lecithin; monoglyceride; phytosterol; protein; saponin extract; polyethylene glycol; propanediol; lecithin extracted from egg yolks; lecithin extracted from seafoods; lecithin extracted from soybeans; lecithin extracted from milk; lecithin extracted from rapeseed; lecithin extracted from cottonseed; or sunflower oil.

Embodiments of the composition may include, for example, concentrations of water as follows: from about 50 wt % to about 95 wt %; from about 55 wt % to about 95 wt %; from about 60 wt % to about 95 wt %; from about 65 wt % to about 95 wt %; from about 70 wt % to about 95 wt %; from about 75 wt % to about 95 wt %; from about 80 wt % to about 95 wt %; from about 50 wt % to about 90 wt %; from about 50 wt % to about 85 wt %; from about 50 wt % to about 80 wt %; from about 50 wt % to about 75 wt %; or from about 50 wt % to about 70 wt %. In some embodiments, the composition is dehydrated to a dry form, and in these embodiments, the composition includes no water or only negligible amounts of water.

Aqueous embodiments of the composition may include, for example, concentrations of adenosine triphosphate as follows: from about 5 wt % to about 30 wt %; from about 8 wt % to about 30 wt %; from about 10 wt % to about 30 wt %; from about 13 wt % to about 30 wt %; from about 15 wt % to about 30 wt %; from about 18 wt % to about 30 wt %; from about 20 wt % to about 30 wt %; from about 23 wt % to about 30 wt %; from about 25 wt % to about 30 wt %; from about 5 wt % to about 28 wt %; from about 5 wt % to about 25 wt %; from about 5 wt % to about 23 wt %; from about 5 wt % to about 20 wt %; from about 5 wt % to about 18 wt %; or from about 5 wt % to about 15 wt %.

Dry embodiments of the composition may include, for example, concentrations of adenosine triphosphate as follows: from about 50 wt % to about 95 wt %; from about 55 wt % to about 95 wt %; from about 60 wt % to about 95 wt %; from about 65 wt % to about 95 wt %; from about 70 wt % to about 95 wt %; from about 75 wt % to about 95 wt %; from about 80 wt % to about 95 wt %; from about 50 wt % to about 90 wt %; from about 50 wt % to about 85 wt %; from about 50 wt % to about 80 wt %; from about 50 wt % to about 75 wt %; or from about 50 wt % to about 70 wt %.

Aqueous embodiments of the composition may include, for example, concentrations of adenosine triphosphate disodium salt as follows: from about 5 wt % to about 30 wt %; from about 8 wt % to about 30 wt %; from about 10 wt % to about 30 wt %; from about 13 wt % to about 30 wt %; from about 15 wt % to about 30 wt %; from about 18 wt % to about 30 wt %; from about 20 wt % to about 30 wt %; from about 23 wt % to about 30 wt %; from about 25 wt % to about 30 wt %; from about 5 wt % to about 28 wt %; from about 5 wt % to about 25 wt %; from about 5 wt % to about 23 wt %; from about 5 wt % to about 20 wt %; from about 5 wt % to about 18 wt %; or from about 5 wt % to about 15 wt %.

Dry embodiments of the composition may include, for example, concentrations of adenosine triphosphate disodium salt as follows: from about 50 wt % to about 95 wt %; from about 55 wt % to about 95 wt %; from about 60 wt % to about 95 wt %; from about 65 wt % to about 95 wt %; from about 70 wt % to about 95 wt %; from about 75 wt % to about 95 wt %; from about 80 wt % to about 95 wt %; from about 50 wt % to about 90 wt %; from about 50 wt % to about 85 wt %; from about 50 wt % to about 80 wt %; from about 50 wt % to about 75 wt %; or from about 50 wt % to about 70 wt %.

Aqueous embodiments of the composition may include, for example, concentrations of pectin as follows: from about 0.5 wt % to about 10 wt %; from about 0.8 wt % to about 10 wt %; from about 1.0 wt % to about 10 wt %; from about 1.3 wt % to about 10 wt %; from about 1.5 wt % to about 10 wt %; from about 1.8 wt % to about 10 wt %; from about 2.0 wt % to about 10 wt %; from about 2.3 wt % to about 10 wt %; from about 2.5 wt % to about 10 wt %; from about 2.8 wt % to about 10 wt %; from about 1.0 wt % to about 9.0 wt %; from about 1.0 wt % to about 8.5 wt %; from about 1.0 wt % to about 8.0 wt %; from about 1.0 wt % to about 7.5 wt %; from about 1.0 wt % to about 7.0 wt %; from about 1.0 wt % to about 6.5 wt %; from about 1.0 wt % to about 6.0 wt %; from about 1.0 wt % to about 5.5 wt %; from about 1.0 wt % to about 5.0 wt %; from about 1.0 wt % to about 4.5 wt %; from about 1.0 wt % to about 4.0 wt %; from about 1.0 wt % to about 3.5 wt %; or from about 1.0 wt % to about 3.0 wt %.

Dry embodiments of the composition may include, for example, concentrations of pectin as follows: from about 5 wt % to about 30 wt %; from about 8 wt % to about 30 wt %; from about 10 wt % to about 30 wt %; from about 13 wt % to about 30 wt %; from about 15 wt % to about 30 wt %; from about 18 wt % to about 30 wt %; from about 20 wt % to about 30 wt %; from about 23 wt % to about 30 wt %; from about 25 wt % to about 30 wt %; from about 5 wt % to about 28 wt %; from about 5 wt % to about 25 wt %; from about 5 wt % to about 23 wt %; from about 5 wt % to about 20 wt %; from about 5 wt % to about 18 wt %; or from about 5 wt % to about 15 wt %; from about 8 wt % to about 15 wt %; or from about 10 wt % to about 15 wt %.

Aqueous embodiments of the composition may include, for example, concentrations of chitosan as follows: from about 0.1 wt % to about 10 wt %; from about 0.2 wt % to about 10 wt %; from about 0.3 wt % to about 10 wt %; from about 0.4 wt % to about 10 wt %; 0.5 wt % to about 10 wt %; from about 0.8 wt % to about 10 wt %; from about 0.4 wt % to about 10 wt %; from about 1.3 wt % to about 10 wt %; from about 1.5 wt % to about 10 wt %; from about 1.8 wt % to about 10 wt %; from about 2.0 wt % to about 10 wt %; from about 2.3 wt % to about 10 wt %; from about 2.5 wt % to about 10 wt %; from about 2.8 wt % to about 10 wt %; from about 0.4 wt % to about 9.0 wt %; from about 0.4 wt % to about 8.5 wt %; from about 0.4 wt % to about 8.0 wt %; from about 0.4 wt % to about 7.5 wt %; from about 0.4 wt % to about 7.0 wt %; from about 0.4 wt % to about 6.5 wt %; from about 0.4 wt % to about 6.0 wt %; from about 0.4 wt % to about 5.5 wt %; from about 0.4 wt % to about 5.0 wt %; from about 0.4 wt % to about 4.5 wt %; from about 0.4 wt % to about 4.0 wt %; from about 0.4 wt % to about 3.5 wt %; or from about 0.4 wt % to about 3.0 wt %.

Dry embodiments of the composition may include, for example, concentrations of chitosan as follows: 0.5 wt % to about 10 wt %; from about 0.8 wt % to about 10 wt %; from about 1.0 wt % to about 10 wt %; from about 1.3 wt % to about 10 wt %; from about 1.5 wt % to about 10 wt %; from about 1.8 wt % to about 10 wt %; from about 2.0 wt % to about 10 wt %; from about 2.3 wt % to about 10 wt %; from about 2.5 wt % to about 10 wt %; from about 2.8 wt % to about 10 wt %; from about 1.0 wt % to about 9.0 wt %; from about 1.0 wt % to about 8.5 wt %; from about 1.0 wt % to about 8.0 wt %; from about 1.0 wt % to about 7.5 wt %; from about 1.0 wt % to about 7.0 wt %; from about 1.0 wt % to about 6.5 wt %; from about 1.0 wt % to about 6.0 wt %; from about 1.0 wt % to about 5.5 wt %; from about 1.0 wt % to about 5.0 wt %; from about 1.0 wt % to about 4.5 wt %; from about 1.0 wt % to about 4.0 wt %; from about 1.0 wt % to about 3.5 wt %; or from about 1.0 wt % to about 3.0 wt %.

Aqueous embodiments of the composition may include, for example, concentrations of calcium chloride as follows: from about 0.05 wt % to about 3.0 wt %; wt % to about 3.0 wt %; from about 0.1 wt % to about 3.0 wt %; from about 0.15 wt % to about 3.0 wt %; from about 0.20 wt % to about 3.0 wt %; from about 0.25 wt % to about 3.0 wt %; from about 0.30 wt % to about 3.0 wt %; from about 0.35 wt % to about 3.0 wt %; from about 0.40 wt % to about 3.0 wt %; from about 0.45 wt % to about 3.0 wt %; from about 0.50 wt % to about 3.0 wt %; from about 0.10 wt % to about 2.5 wt %; from about 0.10 wt % to about 2.0 wt %; from about 0.10 wt % to about 1.5 wt %; from about 0.10 wt % to about 1.3 wt %; from about 0.10 wt % to about 1.0 wt %; or from about 0.10 wt % to about 0.80 wt %.

Dry embodiments of the composition may include, for example, concentrations of calcium chloride as follows: 0.5 wt % to about 10 wt %; from about 0.8 wt % to about 10 wt %; from about 1.0 wt % to about 10 wt %; from about 1.3 wt % to about 10 wt %; from about 1.5 wt % to about 10 wt %; from about 1.8 wt % to about 10 wt %; from about 2.0 wt % to about 10 wt %; from about 2.3 wt % to about 10 wt %; from about 2.5 wt % to about 10 wt %; from about 2.8 wt % to about 10 wt %; from about 1.0 wt % to about 9.0 wt %; from about 1.0 wt % to about 8.5 wt %; from about 1.0 wt % to about 8.0 wt %; from about 1.0 wt % to about 7.5 wt %; from about 1.0 wt % to about 7.0 wt %; from about 1.0 wt % to about 6.5 wt %; from about 1.0 wt % to about 6.0 wt %; from about 1.0 wt % to about 5.5 wt %; from about 1.0 wt % to about 5.0 wt %; from about 1.0 wt % to about 4.5 wt %; from about 1.0 wt % to about 4.0 wt %; from about 1.0 wt % to about 3.5 wt %; or from about 1.0 wt % to about 3.0 wt %.

The foregoing percentages, concentrations, and ratios are presented by example only and are not intended to be exhaustive or to limit the disclose to the precise percentages, concentrations, and ratios disclosed. It should be appreciated that each value that falls within a disclosed range is disclosed as if it were individually disclosed as set forth herein. For example, a range indicating a weight percentage from about 8 wt % to about 14 wt % additionally includes ranges beginning or ending with all values within that range, including, for example, a range beginning at 8.1 wt %, 8.2 wt %, 9 wt %, 10 wt %, and so forth.

Also, according to one or more non-limiting embodiments of the disclosure, any of the concentrations for ingredients for a combination of the ingredient discussed herein, may indicate the concentration for other ingredients listed above.

Example 1 is a composition. The composition includes a microencapsulation matrix comprising a polysaccharide. The composition includes a plurality of bioactive molecules disposed within the microencapsulation matrix. The plurality of bioactive molecules comprises one or more of adenosine triphosphate (ATP) or adenosine triphosphate (ATP) disodium salt.

Example 2 is a composition as in Example 1, further comprising a shell disposed around the microencapsulation matrix to form a coacervation microencapsulate.

Example 3 is a composition as in any of Examples 1-2, wherein the shell is constructed of one or more of a surfactant or a polysaccharide.

Example 4 is a composition as in any of Examples 1-3, further comprising water, wherein the microencapsulation matrix prevents at least a portion of the plurality of bioactive molecules from undergoing a hydrolysis reaction with the water.

Example 5 is a composition as in any of Examples 1-4, further comprising water, wherein the composition comprises from about 70 wt % to about 90 wt % the water.

Example 6 is a composition as in any of Examples 1-5, wherein the plurality of bioactive molecules comprises the ATP disodium salt; and wherein the composition comprises from about 10 wt % to about 18 wt % the ATP disodium salt.

Example 7 is a composition as in any of Examples 1-6, wherein the composition comprises from about 0.5 wt % to about 3.5 wt % the microencapsulation matrix.

Example 8 is a composition as in any of Examples 1-7, further comprising a surfactant matrix that is constructed of a surfactant, wherein the composition comprises from about 0.4 wt % to about 3.5 wt % the surfactant matrix.

Example 9 is a composition as in any of Examples 1-8, further comprising an effective amount of an inorganic mineral for effectuating a desired result in a human or animal, wherein the composition comprises from about 0.1 wt % to about 1.0 wt % the inorganic mineral.

Example 10 is a composition as in any of Examples 1-9, wherein the effective amount of the inorganic mineral comprises one or more of calcium, magnesium, chloride, phosphate, potassium, or sodium.

Example 11 is a composition as in any of Examples 1-10, further comprising water, wherein the microencapsulation matrix prevents at least a portion of the plurality of bioactive molecules from undergoing a hydrolysis reaction with the water, and wherein the composition further comprises one or more of: a natural or artificial flavoring additive; a natural or artificial coloring additive; or a preservative.

Example 12 is a composition as in any of Examples 1-11, wherein the composition comprises only food-grade components such that the composition is prepared for consumption by a mammalian body.

Example 13 is a composition as in any of Examples 1-12, wherein the composition comprises an effective amount of the plurality of bioactive molecules for increasing a strength of muscle contractions in the mammalian body.

Example 14 is a composition as in any of Examples 1-13, wherein the composition comprises an effective amount of the plurality of bioactive molecules for increasing a speed of cellular metabolism in the mammalian body.

Example 15 is a composition as in any of Examples 1-14, wherein the composition is prepared in a dry powder form.

Example 16 is a composition as in any of Examples 1-15, wherein the plurality of bioactive molecules comprises the ATP disodium salt, and wherein the composition comprises from about 50 wt % to about 90 wt % the ATP disodium salt.

Example 17 is a composition as in any of Examples 1-16, wherein the composition comprises from about 2.5 wt % to about 17.7 wt % the microencapsulation matrix.

Example 18 is a composition as in any of Examples 1-17, further comprising a surfactant matrix configured to be disposed around the microencapsulation matrix, and wherein the composition comprises from about 2 wt % to about 17.7 wt % the surfactant matrix.

Example 19 is a composition as in any of Examples 1-18, wherein the microencapsulation matrix is constructed of one or more of cellulose, glycogen, chitin, pectin, or chitosan.

Example 20 is a composition as in any of Examples 1-19, further comprising a surfactant matrix configured to be disposed around the microencapsulation matrix, and wherein the surfactant matrix is constructed of one or more of: polyethylene glycol; propylene glycol; propanediol; or a lecithin extracted from one or more of egg yolks, seafood, soybeans, milk, rapeseed, cottonseed, or sunflower oil.

Example 21 is a method for preparing a composition that includes an organic nucleotide. The method includes preparing a solution comprising water, a surfactant, a polysaccharide, and the organic nucleotide. The method includes agitating the solution. The method includes processing the solution under shear mixing.

Example 22 is a method as in Example 21, wherein each of the water, the surfactant, the polysaccharide, and the organic nucleotide are intermixed in a colloidal dispersion; and wherein the colloidal dispersion enables increased shelf stability of the organic nucleotide.

Example 23 is a method as in any of Examples 21-22, wherein the organic nucleotide comprises one or more of adenosine triphosphate (ATP) or adenosine triphosphate (ATP) disodium salt.

Example 24 is a method as in any of Examples 21-23, further comprising dehydrating the solution to generate a dry powder comprising the organic nucleotide encapsulated by a microencapsulation matrix.

Example 25 is a method as in any of Examples 21-24, wherein the polysaccharide forms a microencapsulation matrix; and wherein the organic nucleotide is disposed within the microencapsulation matrix such that the microencapsulation matrix prevents the organic nucleotide from undergoing a hydrolysis reaction with the water.

Example 26 is a method as in any of Examples 21-25, further comprising preparing the solution for human consumption, wherein preparing the solution for human consumption comprises adding one or more of: a natural or artificial flavoring additive; a natural or artificial coloring additive; or a preservative.

Example 27 is a method as in any of Examples 21-26, wherein the surfactant comprises one or more of: polyethylene glycol; propylene glycol; propanediol; or a lecithin extracted from one or more of egg yolks, seafood, soybeans, milk, rapeseed, cottonseed, or sunflower oil.

Example 28 is a method as in any of Examples 21-27, wherein the polysaccharide comprises one or more of cellulose, glycogen, chitin, pectin, or chitosan.

Example 29 is a method as in any of Examples 21-28, wherein the polysaccharide comprises a degree of esterification of up to 72% and further comprises a degree of acetylation of up to 30%.

Example 30 is a method as in any of Examples 21-29, wherein the solution comprises from about 70 wt % to about 90 wt % the water.

Example 31 is a method as in any of Examples 21-30, wherein the method results generating in a microencapsulate comprising: a surfactant matrix; a polysaccharide matrix disposed within a shelf formed by the surfactant matrix; and the organic nucleotide within the polysaccharide matrix; wherein the surfactant matrix and the polysaccharide matrix prevent the organic nucleotide from undergoing a hydrolysis reaction with the water.

Example 32 is a method as in any of Examples 21-31, wherein the composition comprises from about 0.5 wt % to about 3.5 wt % the polysaccharide matrix.

Example 33 is a method as in any of Examples 21-32, wherein the composition comprises from about 0.4 wt % to about 3.5 wt % the surfactant matrix.

Example 34 is a method as in any of Examples 21-33, wherein the composition comprises from about 10 wt % to about 18 wt % the organic nucleotide, and wherein the organic nucleotide comprises one or more of ATP or ATP disodium salt.

Example 35 is a method as in any of Examples 21-34, further comprising dehydrating the solution to generate a powder comprising dry microencapsulate, wherein the dry microencapsulate comprises: a surfactant matrix; a polysaccharide matrix disposed within a shelf formed by the surfactant matrix; and the organic nucleotide within the polysaccharide matrix.

Example 36 is a method as in any of Examples 21-35, wherein the powder comprises from about 50 wt % to about 90 wt % the organic nucleotide, and wherein the organic nucleotide comprises one or more of ATP or ATP disodium salt.

Example 37 is a method as in any of Examples 21-36, wherein the powder comprises from about 2.5 wt % to about 17.7 wt % the polysaccharide matrix.

Example 38 is a method as in any of Examples 21-37, wherein the powder comprises from about 2.0 wt % to about 17.7 wt % the surfactant matrix.

Example 39 is a method as in any of Examples 21-38, wherein preparing the solution comprises further adding an effective amount of a mineral for effectuating a desired result in a human, wherein the mineral comprises one or more of calcium, magnesium, chloride, phosphate, potassium, or sodium.

Example 40 is a method as in any of Examples 21-39, wherein preparing the solution comprises adding an effective amount of the organic nucleotide for effectuating a desired result in a human body, wherein the desired result comprises one or more of: increasing a strength of muscle contractions in the mammalian body; or increasing a speed of cellular metabolism in the mammalian body.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims, if any.

Other embodiments will be apparent to those skilled in the art from consideration of the specification and practice of the disclosure disclosed herein. It is intended that the specification and examples be considered as exemplary only, with a true scope and spirit of the invention being indicated by the following exemplary embodiments.

1. A composition comprising:
a microencapsulation matrix comprising a polysaccharide;
a plurality of bioactive molecules disposed within the microencapsulation matrix;
wherein the plurality of bioactive molecules comprises one or more of adenosine triphosphate (ATP) or adenosine triphosphate (ATP) disodium salt.

2. The composition of claim 1, wherein the composition further comprises a shell disposed around the microencapsulation matrix to form a coacervation microencapsulate.

3. The composition of claim 2, wherein the shell is constructed of one or more of a surfactant or a polysaccharide.

4. The composition of claim 1, wherein the composition further comprises water, wherein the microencapsulation matrix prevents at least a portion of the plurality of bioactive molecules from undergoing a hydrolysis reaction with the water.

5. The composition of claim 1, wherein the composition further comprises water, wherein the composition comprises from about 70 wt % to about 90 wt % of water.

6. The composition of claim 1, wherein the plurality of bioactive molecules comprises the ATP disodium salt; and
wherein the composition comprises from about 10 wt % to about 18 wt % the ATP disodium salt.

7. The composition of claim 1, wherein the composition comprises from about 0.5 wt % to about 3.5 wt % of the microencapsulation matrix.

8. The composition of claim 1, wherein the composition further comprises a surfactant matrix that is constructed of a surfactant, wherein the composition comprises from about 0.4 wt % to about 3.5 wt % of the surfactant matrix.

9. The composition of claim 1, wherein the composition further comprises an effective amount of an inorganic mineral for effectuating a desired result in a human or animal, wherein the composition comprises from about 0.1 wt % to about 1.0 wt % of the inorganic mineral.

10. The composition of claim 9, wherein the effective amount of the inorganic mineral comprises one or more of calcium, magnesium, chloride, phosphate, potassium, or sodium.

11. The composition of claim 1, wherein the composition further comprises water, wherein the microencapsulation matrix prevents at least a portion of the plurality of bioactive molecules from undergoing a hydrolysis reaction with the water, and wherein the composition further comprises one or more of:
a natural or artificial flavoring additive;
a natural or artificial coloring additive; or
a preservative.

12. The composition of claim 11, wherein the composition comprises only food-grade components such that the composition is prepared for consumption by a mammalian body.

13. The composition of claim 11, wherein the composition comprises an effective amount of the plurality of bioactive molecules for increasing a strength of muscle contractions in the mammalian body.

14. The composition of claim 11, wherein the composition comprises an effective amount of the plurality of bioactive molecules for increasing a speed of cellular metabolism in the mammalian body.

15. The composition of claim 1, wherein the composition is prepared in a dry powder form.

16. The composition of claim 15, wherein the plurality of bioactive molecules comprises the ATP disodium salt, and wherein the composition comprises from about 50 wt % to about 90 wt % of the ATP disodium salt.

17. The composition of claim 15, wherein the composition comprises from about 2.5 wt % to about 17.7 wt % the microencapsulation matrix.

18. The composition of claim 15, wherein the composition further comprises a surfactant matrix configured to be disposed around the microencapsulation matrix, and wherein the composition comprises from about 2 wt % to about 17.7 wt % of the surfactant matrix.

19. The composition of claim 1, wherein the microencapsulation matrix is constructed of one or more of cellulose, glycogen, chitin, pectin, or chitosan.

20. The composition of claim 1, wherein the composition further comprises a surfactant matrix configured to be disposed around the microencapsulation matrix, and wherein the surfactant matrix is constructed of one or more of:
polyethylene glycol;
propylene glycol;
propanediol; or
a lecithin extracted from one or more of egg yolks, seafood, soybeans, milk, rapeseed, cottonseed, or sunflower oil.

21-40. (canceled)
